SEQUENCE LISTING (2 sequences)

SEQ ID NO: 1 - DNA, 22 bp, Artificial
  misc_feature (1)..(22): OLIGONUCLEOTIDE HAVING A FLUORESCENT LABEL (Cy 5-dCTP) AT ITS 5′-TERMINAL.
ctagtctgtgaagtgtctgatc

SEQ ID NO: 2 - DNA, 22 bp, Artificial
  misc_feature (1)..(22): TARGET OLIGONUCLEOTIDE HAVING Cy5 AT ITS 5′-TERMINAL.
gatcagacacttcacagactag

(19) United States
(12) Patent Application Publication
(10) Pub. No.: US 2004/0096838 A1
(43) Pub. Date: May 20, 2004

(54) DNA chip and its preparation

(76) Inventors: Tadahisa Sato, Kanagawa (JP); Koki Nakamura, Kanagawa (JP); Hiroshi Shinoki, Saitama (JP); Junji Nishigaki, Kanagawa (JP)

Correspondence Address: REED SMITH, LLP, ATTN: PATENT RECORDS DEPARTMENT, 599 LEXINGTON AVENUE, 29TH FLOOR, NEW YORK, NY, 10022-7650, US

(21) Appl. No.: 10/326,350

(22) Filed: Dec. 20, 2002

Related U.S. Application Data
(62) Division of application No. 09/749,714, filed on Dec. 27, 2000, now abandoned.

(30) Foreign Application Priority Data
Dec. 27, 1999 (JP) 11-371072
Dec. 27, 1999 (JP) 11-371330
Dec. 27, 1999 (JP) 11-371331

(51) Int. Cl. C12Q 1/68; C12M 1/34; B05D 3/00

(52) U.S. Cl. 435/6; 435/287.2; 427/2.11

(57) ABSTRACT

An analytical element (typically DNA chip) composed of a solid carrier and a group of nucleotide derivatives or their analogues fixed to the solid carrier via covalent bonding can be favorably prepared by bringing in a liquid phase a group of nucleotide derivatives or their analogues having at one end or its vicinity a reactive group into contact with a solid carrier having on its surface a reactive group, so as to produce between these reactive groups an irreversible addition reaction, an cyclizing addition reaction, or a coupling reaction.

### FIELD OF THE INVENTION

This invention relates to a solid carrier to which nucleotide derivatives or their analogues (e.g., oligonucleotides, polynucleotides, and peptide-nucleotides) are attached, which is generally named DNA chip and which is favorably employable for detecting, with high sensitivity, complementary nucleic acid fragments.

### BACKGROUND OF THE INVENTION

Detection of a nucleic acid fragment is generally performed using a probe oligonucleotide which is complementary to the nucleic acid fragment to be detected, by way of hybridization. The probe oligonucleotide is generally fixed onto a solid carrier (e.g., solid substrate) to produce a so-called DNA chip. In the detection procedures, a nucleic acid fragment in a sample liquid is provided with a fluorescent label or a radioisotope label, and then the sample liquid is brought into contact with the probe oligonucleotide of the DNA chip. If the labelled nucleic acid fragment in the sample liquid is complementary to the probe oligonucleotide, the labelled nucleic acid fragment is combined with the probe oligonucleotide by hybridization. The labelled nucleic acid fragment fixed to the DNA chip by hybridization with the probe oligonucleotide is then detected by an appropriate detection method such as fluorometry or autoradiography. The DNA chip is widely employed in the gene technology, for instance, for detecting a complementary nucleic acid fragment and sequencing the detected nucleic acid fragment.

The DNA chip can be utilized to efficiently detect a large number of complementary nucleic acid fragments in a small amount of a sample liquid within a short period of time.

Detection of nucleic acid fragment using an electrochemical label is also known (Japanese Patent Provisional Publication No. 9-288080, and a preprint of the 57th Analytical Chemistry Conference pp. 137-138 (1996)).

P. E. Nielsen et al., Science, 254, 1497-1500(1991) and P. E. Nielsen et al., Biochemistry, 36, pp.5072-5077 (1997) describe PNA (Peptide Nucleic Acid or Polyamide Nucleic Acid) which has no negative charge and functions in the same manner as DNA fragment does. PNA has a polyamide skeleton of N-(2-aminoethyl)glycine units and has neither glucose units nor phosphate groups.

Since PNA is electrically neutral and is not charged in the absence of an electrolytic salt, PNA is able to hybridize with a complementary nucleic acid fragment to form a hybrid which is more stable than the hybrid structure given by a probe oligonucleotide and its complementary nucleic acid fragment (Preprint of the 74th Spring Conference of Japan Chemical Society, pp. 1287, reported by Naomi Sugimoto).

Japanese Patent Provisional Publication No.11-332595 describes a PNA probe fixed onto a solid carrier at its one end and a detection method utilizing the PNA probe. The PNA probe is fixed onto the solid carrier by the known combination of avidin and biotin.

The aforementioned P. E. Nielsen et al., Science, 254, 1497-1500(1991) also describes a PNA probe labelled with an isotope element and a detection method of a complementary nucleic acid fragment.

Since the PNA probe shows no electric repulsion to a target nucleic acid fragment in a sample liquid, an improved high detection sensitivity is expected.

At present, two methods are known for preparing a DNA chip having a solid carrier and oligonucleotides or polynucleotides fixed onto the carrier. One preparation method comprises preparing oligonucleotides or polynucleotides, step by step on the carrier. This method is named “on-chip method”. A typical on-chip method is described in Foder, S. P. A., Science, 251, page 767 (1991).

Another preparation method comprises fixing separately prepared oligonucleotides or polynucleotides onto a solid carrier. Various methods are known for various oligonucleotides and polynucleotides.

In the case of the complementary nucleotide derivatives (which are synthesized using mRNA as mold) or PCR products (which are DNA fragments prepared by multiplying cDNA by PCR method), an aqueous solution of the prepared DNA fragment is spotted onto a solid carrier having a poly-cationic coat in a DNA chip-preparing device to fix the DNA fragment to the carrier via electrostatic bonding, and then blocking a free surface of the polycationic coat.

In the case that the oligonucleotides are synthetically prepared and have a functional group, an aqueous solution of the synthetic oligonucdeotides is spotted onto an activated or reactive solid carrier to produce covalent bonding between the oligonucleotides and the carrier surface. See Lamture, J. B., et al., Nucl. Acids Res., 22, 2121-2125, 1994, and Guo, Z., et al., Nucl. Acids Res., 22, 5456-5465, 1994. Generally, the oligonucleotides are covalently bonded to the surface activated carrier via linking groups.

Also known is a process comprising the steps of aligning small polyacrylamide gels on a glass plate and fixing synthetic oligonucleotides onto the glass plate by making a covalent bond between the polyacrylamide and the oligonucleotide (Yershov, G., et al., Proc. Natl. Acad. Sci. USA, 94, 4913(1996)). Sosnowski, R. G., et al., Proc. Natl. Acad. Sci. USA, 94, 1119-1123 (1997) discloses a process comprising the steps of placing an array of microelectrodes on a silica chip, forming on the microelectrode a streptoavidin-comprising agarose layer, and attaching biotin-modified DNA fragments to the agarose layer by positively charging the agarose layer. Schena, M., et al., Proc. Natl. Acad. Sci. USA, 93, 10614-10619 (1996) teaches a process comprising the steps of preparing a suspension of an amino group-modified PCR product in SSC (i.e., standard sodium chloride-citric acid buffer solution), spotting the suspension onto a slide glass, incubating the spotted glass slide, treating the incubated slide glass with sodium borohydride, and heating thus treated slide glass.

As is explained above, most of the known methods of fixing separately prepared DNA fragments onto a solid carrier utilize the electrostatic bonding or the covalent bonding such as described above.

In any DNA chips having separately prepared oligonucleotide probes on its solid carrier, the oligonucleotide probes should be firmly fixed onto the carrier, so that the hybridization can proceed smoothly between the fixed oligonucleotide probes and target DNA fragments complementary to the fixed oligonucleotide probes.

Further, it is preferred that a surface area of the solid carrier other than the portion to which the probe oligonucleotides are fixed is inactive to the labelled DNA fragments, so that non-complementary DNA fragments in the liquid sample can be kept from attaching onto the surface in the course of the detection procedure utilizing hybridization and kept from remaining on the surface of the carrier. If the non-complementary DNA fragments remain in the surface of the carrier, the accuracy of the detection decreases.

U.S. Pat. No. 5,387,505 describes a method of separating a target DNA fragment by binding target DNA fragments labelled with a biotin molecule with a substrate having avidin molecules.

U.S. Pat. No. 5,094,962 discloses a detection tool for a ligand-receptor assay in which receptor molecules are bonded to a porous polymer particle having a reactive group.

### SUMMARY OF THE INVENTION

It is an object of the present invention to provide a solid carrier to which a group of nucleotide derivatives or their analogues (e.g., oligonucleotides, polynucleotides, and peptide-nucleotides, which serve as probes for detecting complementary DNA fragments by way of hybridization) are attached and which is favorably employable for detecting, with high sensitivity, complementary nucleic acid fragments.

It is another object of the invention to provide a DNA chip which is employable in the procedure for detecting complementary DNA fragments without performing in advance a blocking procedure, that is, a procedure of inactivating the solid carrier in the areas having no probes, so as to keep non-complementary DNA fragments from fixing on the carrier by non-hybridization mechanism.

The present inventors have discovered that an analytical element (typically DNA chip) composed of a solid carrier and a group of nucleotide derivatives or their analogues fixed to the solid carrier via covalent bonding can be favorably prepared by bringing in a liquid phase a group of nucleotide derivatives or their analogues having at one end or its vicinity a reactive group into contact with a solid carrier having on its surface a reactive group, so as to perform between these reactive groups an irreversible addition reaction, an cyclizing addition reaction, or a coupling reaction.

Accordingly, the present invention resides in an element comprising a solid carrier and a group of nucleotide derivatives or their analogues which are fixed to the solid carrier via covalent bonding, in which the covalent bonding is produced by an irreversible addition reaction, a cyclizing addition reaction, or a coupling reaction between a reactive group attached to the solid carrier and a reactive group attached to the nucleotide derivative or the analogue.

In the irreversible addition reaction, it is preferred that the reactive group attached to the solid carrier contains a quinone moiety and the reactive group attached to the nucleotide derivative or analogue contains an amino moiety or a thiol moiety. More preferably, the quinone moiety is a 1,4-benzophenone moiety or a 1,4-naphthoquinone moiety.

In the cyclizing addition reaction, it is preferred that one of the reactive groups contains a diene moiety and another contains a dienophile moiety. More preferably, the reactive group attached to the nucleotide derivative or analogue contains a diene moiety and the reactive group attached to the solid carrier contains a dienophile moiety.

In the coupling reaction, it is preferred that one reactive group is derived from a compound having the following formula (1):

Z=(CR¹-C²)ₙ═N—R³ (1)

wherein Z is an oxygen atom or a group of R⁴N or R⁴R⁵N⁺A in which each of R⁴ and R⁵ independently is a hydrogen atom, an alkyl group or an aryl group, and A is an inorganic or organic anion; each of R¹ and R² independently is a hydrogen atom or a substituent group; R³ is a hydrogen atom, or a group of NR⁶R⁷, SR⁶, SO₂R⁶, or COR⁶ in which each of R⁶ and R⁷ independently is a hydrogen atom or a substituent group; n is 0, 1, or 2; provided that R¹ and R² can be combined together to form a ring; and at least one of Z, R¹ and R² produces covalent bonding with a surface of the solid carrier or the nucleotide derivative or analogue; and another reactive group is capable of causing a coupling reaction with a nitrogen atom of the reactive group derived from the compound of the formula (1).

In the coupling reaction, it is particularly preferred that the reactive group derived from the formula (1) is attached to the surface of the solid carrier, and the reactive group causing a coupling reaction with a nitrogen atom of the reactive group derived from the formula (1) is attached to the nucleotide derivative or analogue.

The invention further resides in a method for preparing an element comprising a solid carrier and a group of nucleotide derivatives or their analogues fixed to the solid carrier via covalent bonding, which comprises bringing in a liquid phase a group of nucleotide derivatives or their analogues having at one end thereof or its vicinity a reactive group into contact with a solid carrier having thereon a reactive group, to cause between these reactive groups an irreversible addition reaction, a cyclizing addition reaction, or a coupling reaction.

The detection method of the invention for oligonucleotides or polynucleotides such as DNA fragments can be performed by bringing the solid carrier having probes (i.e., a group of nucleotide derivatives or their analogues) fixed onto its surface into contact with oligonucleotides or polynucleotides (such as target DNA fragments) which are complementary to the probes of nucleotide derivatives or their analogues fixed onto the surface of the solid carrier in the presence of an aqueous solvent, so as to combine the complementary oligonucleotides or polynucleotides with the nucleotide derivatives or their analogues.

### BRIEF DESCRIPTION OF DRAWINGS

FIG. 1 schematically illustrates a representative DNA fragment-fixed solid carrier and its production processes according to the invention.

FIG. 2 schematically illustrates a typical process for fixing an oligonucleotide probe onto a solid carrier according to the irreversible addition reaction.

### DETAILED DESCRIPTION OF THE INVENTION

### Solid Carrier

The solid carrier can be any of known solid carriers or their equivalent materials, for instance, a glass plate, a resin plate, a metal plate, a glass plate covered with polymer coat, a glass plate covered with metal coat, and a resin plate covered with metal coat. Also employable is a SPR (surface plasmon resonance) sensor plate which is described in Japanese Patent Provisional Publication No. 11-332595. CCD is also employable as described in Nucleic Acids Research, 1994, Vol.22, No.11, 2124-2125.

The surface of the solid carrier can be coated with a polymer layer or a cross-linked polymer layer.

The solid carrier should have on its surface a plurality of reactive groups which are capable of reacting with a reactive group attached to a probe compound (i.e., nucleotide derivative or its analogue) to produce a covalent bonding between the solid carrier and the probe compound, so as to fix the probe compound onto the surface of the solid carrier. The reaction can proceed according to an irreversible addition reaction mode, a cyclizing addition reaction mode, or a coupling reaction mode.

The irreversible addition reaction means a reaction in which the thermodynamic equilibrium of addition reaction prominently deviates toward the production system. In the irreversible addition reaction according to the invention, a rate of the forward reaction proceeding from the original system (system prior to initiation of the reaction) to the production system is extremely faster than a rate of the reverse reaction proceeding from the production system to the original system, and accordingly the reverse reaction can be ignored. Otherwise, a product of the addition reaction may be further converted into other compound by the following reactions.

There are no specific limitations with respect to the reactive groups participating in the irreversible addition reaction according to the invention. It is preferred, however, that the reactive group attached to the solid carrier contains a quinone moiety (e.g., 1,4-benzoquinone moiety or 1,4-naphthoquinone moiety) and the reactive group attached to the nucleotide derivative or analogue contains an amino moiety or a thiol moiety.

The quinone moiety may have one or more substituents such as an alkyl group having 1 to 6 carbon atoms, a cycloalkyl group having 3 to 6 carbon atoms, an alkenyl group having 2-to 6 carbon atoms, an alkynyl group having 2 to 6 carbon atoms, a cyano group, a halogen atom (e.g., F, Cl, Br), a hydroxyl group, an acyl group having 1 to 6 carbon atoms, an acyloxy group having 1 to 6 carbon atoms, an ester group having 2 to 6 carbon atoms, and an acylamino group having 1 to 6 carbon atoms. The substituent group preferably is an electron-attracting group such as cyano, halogen, or acyloxy.

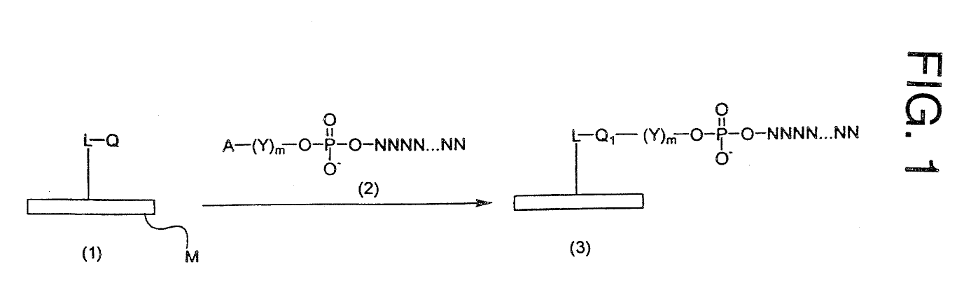

FIG. 1 in the attached drawings schematically shows progress of the irreversible addition reaction adopted in the invention. The chain molecule (LQ) is composed of a quinone moiety (Q) placed on the free end and a linking group (L) which connects the quinone moiety (Q) with the surface of the solid carrier (M). The solid carrier (1) having on its surface the chain molecule (LQ) is brought into contact with a DNA fragment (2) having at its terminal an amino group or a thiol group (A), to give a DNA chip (3). In FIG. 1, Y represents a cross linker, such as a single bond, an alkylene group (e.g., hexylene group), or an N-alkylaminoalkylene group (e.g., N-methylaminoalkylene group). m is 0 or 1, and Q¹ is a group determined by the mode of reaction between Q and A. The chain composed of NNNNNN . . . NN and phosphoric acid moiety represents a DNA fragment.

For fixing the DNA fragment or other nucleotide derivative or its analogue onto the solid carrier by the irreversible addition reaction, the surface of the solid carrier is preferably coated with a polycationic compound such as poly-L-lysine, polyethyleneimide, or polyalkylamine, or treated with a silane coupling agent such as γ-aminopropyltriethoxysilane, N-β(aminoethyl)-γ-aminopropyl, trimethoxysilane, or N-β(aminoethyl)-γ-aminopropylmethyldimethoxysilane, so as to place amino groups on the surface of the solid carrier.

In place of the amino group, an aldehyde group, an epoxy group, a carboxyl group, a hydroxyl group, or a thiol group can be placed on the solid carrier.

The reactive group containing a quinone moiety (Q) can be placed on the surface of the solid carrier, by the steps of reacting 1,4-benzoquinone or 1,4-naphthoquinone with a mercaptoalkanoic acid [according to the method described in Indian Journal of Chemistry, 15B, 970(1997)] to attach a carboxyl group to the quinone derivative; and bringing the carboxyl group-attached quinone derivative, in the presence of a carboxyl group-activating reagent, into contact with the amino group attached to the surface of the solid carrier. Examples of the carboxyl group-activating reagents include known carbodiimides such as DDC and WSC, carbonate esters, and chlorides such as thionyl chloride and oxalyl chloride.

In the reaction using the carboxyl group-activating reagent, a base such an organic base or an inorganic base can be employed. Preferred examples of the bases include 1-methyl-2-pyrrolidone, triethylamine, pyridine, potassium carbonate, and sodium carbonate.

The cyclizing addition reaction (i.e., cycloaddition reaction) can be represented by the Diels-Alder reaction, [4+2]-cyclizing-reaction, ene reaction, 1,3-dipolecyclizing addition reaction, and [2+2]-cyclizing reaction. These reactions are performed using two reactants (reacting components) which are extremely specific to each other. Accordingly, the reactants possibly remaining on the surface of the solid carrier after the fixing the probe compounds having a counter reactant are inactive to fix contaminant DNA fragments on the surface in the course of the detection procedure utilizing hybridization. The fixation of contaminant DNA fragments onto the solid carrier causes increase a background value in the measurement such as fluorometry.

The reactants of the above-mentioned cyclizing addition reactions are already known and can be chosen from the descriptions of the following texts: Carruthers, “Cycloaddition Reactions in Organic Synthesis”, Permon Press, 1990; Dale L. Boger, Steven N. Weinreb, “Hetero Diels-Alder Methodology in Organic Synthesis”, Academic Press, Inc., 1987; paul A. Grieco, “Organic Synthesis in Water”, Blackie Academic & Professional, 1998.

Various pairs of the reactants preferably employed in the invention are described in “Organic Reactions”, John Wiley & Sons, Inc., Vol. 4, Chapters 1 & 2, Vol. 5, Chapter 3; Japanese Patent Provisional Publications No. 6-64343, No. 7-132685, and No. 7-144478.

The cyclizing addition reaction generally can proceed at an ambient temperature or a lower temperature. Preferred are, however, the reactions which can proceed at a temperature from room temperature to 180° C. The reaction preferably proceeds at a temperature of lower than 120° C.

The cyclizing addition reaction can be performed in the absence of water. However, in order to well disperse the probe compound (e.g., oligonucleotide) in the fixing procedure, water is preferably employed. Therefore, it is preferred that the reactants are resistant to water.

The cyclizing addition reaction may be accelerated in the presence of a catalyst such as zinc chloride, tin tetrachloride, or magnesium bromide. Therefore, the fixation of the probe compound onto the surface of the solid carrier can be performed in the presence of the catalyst.

A reactant of the cyclizing addition reaction can be attached to the surface of the solid carrier by bringing the carrier into contact with a silane coupling agent containing the reactant moiety. The reactant can be temporarily protected and removed in the fixing procedure.

In the case of utilizing a solid carrier having amino groups on its surface, a compound containing the reactant moiety and a linking group reactive with the amino group to produce a covalent bonding can be employed. Examples of the linking groups include carboxyl, formyl, sulfo, isocyanato, isothiocyanato, and acid anhydride, Preferred are a combination of a dienophile such as maleic anhydride attached to the solid carrier with an appropriate diene compound attached to the probe compound to produce a maleimido bonding.

The surface of the solid carrier can have a polymer containing a reactive moiety which can produce a covalent bonding upon a cyclizing addition reaction. This polymer may has a recurring unit derived from a silane-coupling agent. The polymer can be coated on the solid carrier in the form of latex, which may be heated on the carrier.

If the reactant has an acrylate moiety or an acrylamido moiety, a multi-functional polymer containing unreacted moieties can be used.

If the reactant has an amino moiety or-an aliphatic hydroxyl moiety, it can be fixed onto the solid carrier which is previously treated with a multi-functional coupling agent such as a silane coupling agent, a multi-functional vinyl sulfone, or cyanyl chloride. Examples of the silane-coupling agents are described hereinbefore.

The reaction for fixing the reactant onto the solid carrier can be performed under the conditions similar to those employed in the irreversible addition reactions.

The coupling reaction which can be employed in the invention for fixing a probe compound onto a surface of a solid carrier is essentially the same as the coupling reaction known in the art of silver halide photographic material which proceeds between a color-developing oxidation compound [which is represented by the formula (1)] and a coupler. The coupling agent (i.e., coupler) can be an aromatic alkyl compound such as aniline or an arylsulfinic acid. Other coupling agents are described in Japanese Patent Provisional Publication No. 10-207028.

In the present invention, it is preferred that the reactive moiety of the formula (1) is attached to the solid carrier and that the coupling moiety is attached to the probe compound such as oligonucleotide.

Attachment of the reactive moiety of the formula (1) or the coupling moiety to the solid carrier can be performed almost in the same manner as described hereinbefore in the procedures for the irreversible addition reaction and the cyclizing addition reaction.

### Probe Compound—Nucleotide Derivative or Its Analogue

A probe compound (which is a nucleotide derivative or its analogue, such as polynucleotide, oligonucleotide, PNA, or one of their analogues) having at its one terminal a reactive group which is other reactive component of the irreversible addition reaction, cyclizing addition reaction, and coupling reaction to form a covalent bonding can be prepared by one of the following two methods.

(1) A primer which is a probe compound having an appropriate reactive group is multiplied by the PCR method.

(2) A primer which is a probe compound having a reactive group such as amino is multiplied by the PCR method, and to the resulting probe compounds having a reactive group is attached an appropriate reactive group.

Generally, the latter method can be readily performed, and accordingly is preferred in the present invention. The attachment of an amino group to the probe compound can be attained by forming an amide bonding between the amino group and a carboxyl group of an appropriate compound using an appropriate condensing agent.

In more detail, the probe compound having an amino group at its terminal can be reacted with a compound having as its partial structure a reactive group such as 1,2-diol, 1,3-diol, 1,2-aminoalcohol, 1,3-aminoalcohol, 1,2-diamine, 2-hydroxycarboxylic acid, 2-aminocarboxylic acid, 3-hydroxycarboxylic acid or 3-amiocarboxylic acid, to combine the structure of reactive compound with the probe compound. A probe compound having a thiol group at is terminal also can be favorably employed.

The probe compounds, namely, nucleotide derivatives or their analogues to be fixed to the solid carrier can be. oligonucleotides, polynucleotides, or peptide-nucleotides. A DNA fragment can be employed as the probe compound.

The nucleotide derivative may be polynucleotide such as cDNA, a portion of cDNA, or EST. The polynucleotide is favorably employed for studying gene expression. Otherwise, nucleotide derivatives to be fixed onto the solid carrier may be oligonucleotides, which are favorably employed for studying variations and polymorphism of gene. The oligonucleotide to be fixed onto the solid carrier preferably is one of 3 to 50-mers, more preferably 10 to 25 mers. The oligonucleotide and polynucleotide can have one or more substituent groups and/or cross-linking groups, provided that the attachment of these groups does not impart adverse influence to the function of the oligonucleotide and polynucleotide. For instance, INA (locked nucleic acid) which is described in J. Am. Chem. Soc., 1998, 120, 13252-13253, can be employed.

### Procedure of Fixing Probe Compounds

The nucleotide derivatives (or their analogues) to be fixed on the solid carrier are dissolved or dispersed in an aqueous solution. Generally, the aqueous solution is once placed on a polymer plate having 96 or 384 wells, and then spotted onto a solid carrier using a spotting means.

The reaction for fixing the probe compounds having at their terminal (or in the vicinity) one reactive moiety onto the solid carrier having a counter reactive moiety can be performed at ambient temperatures or under cooling (such as 5 to 10° C.) or heating. The heating condition is favorably adopted. Preferably, the reaction is performed at 4 to 150° C., more preferably at 50 to 130° C., most preferably at 50 to 100° C. The reaction can be conducted in a pressure-resistant vessel such as an autoclave.

In order to keep the spotted aqueous solution from evaporating, it is preferred to add a high boiling-point compound to the aqueous solution containing nucleotide derivatives. The high boiling-point compound should be soluble in an aqueous medium, should not disturb hybridization procedure, and preferably has an appropriate viscosity. Examples of the high boiling-point compounds include glycerol, ethylene glycol, dimethylsulfoxide, and a hydrophilic polymer having a low molecular weight (typically, in the range of 10³ to 10⁶) such as polyacrylamide, polyethylene glycol, or poly(sodium acrylate). The high boiling-point compound preferably is glycerol or ethylene glycol. The high boiling-point compound is preferably incorporated into an aqueous nucleotide derivative solution in an amount of 0.1 to 2 vol. %, particularly 0.5 to 1 vol. %. Otherwise, the spotted aqueous solution is preferably kept at under the conditions of a high humidity (such as 90% RH or more) and an ordinary temperature (25 to 50° C.).

The aqueous solution is spotted onto the solid carrier under the condition that each drop of the solution generally has a volume of 100 pL to 1 μL, preferably 1 to 100 nL. The nucleotide derivatives preferably spotted onto the solid carrier are in an amount (number) of 10² to 10⁵/cm². In terms of mol., 1 to 10⁻¹⁵ moles are spotted. In terms of weight, several ng or less of nucleotide derivatives are spotted. The spotting of the aqueous solution is made onto the solid carrier to form several dots having almost the same shape and size. It is important to prepare these dots to have the same shape and size, if the hybridization is quantitatively analyzed. Several dots are formed separately from each other with a distance of 1.5 mm or less, preferably 100 to 300 μm. One dot preferably has a diameter of 50 to 300 μm.

After the aqueous solution is spotted on the solid carrier, the spotted solution is preferably incubated, namely, kept for a certain period at room temperature or under warming, so as to fix the spotted nucleotide derivatives onto the carrier. In the course of incubation, UV irradiation or surface treatment using sodium borohydride or a Shiff reagent may be applied. The UV irradiation under heating is preferably adopted. It is assumed that these treatments are effective to produce additional linkage or bonding between the solid carrier and the attached oligonucleotide derivatives. The free (namely, unfixed) nucleotide derivatives are washed out using an aqueous solution. Thus washed solid carrier is then dried to give a nucleotide derivative-fixed solid carrier (such as DNA chip) of the invention.

It is not necessary to subject thus prepared analytical element to blocking treatment. However, the analytical element may be subjected to blocking treatment, if desired.

The nucleotide derivative-fixed solid carrier of the invention is favorably employable for monitoring of gene expression, sequencing of base arrangement of DNA, analysis of mutation, analysis of polymorphism, by way of hybridization.

### Sample Nucleic Acid Fragment—Target

A target DNA fragment or a sample DNA fragment, which is subjected to the analysis concerning the presence of a complementary DNA fragment can be obtained from various origins. In the analysis of gene, the target DNA fragment is prepared from a cell or tissue of eucaryote. In the analysis of genome, the target DNA fragment is obtained from tissues other than erythrocyte. In the analysis of mRNA, the target sample is obtained from tissues in which mRNA is expressed. If the DNA chip has an oligonucleotide fixed in its solid carrier, the target DNA fragment preferably has a low molecular weight. The target DNA may be multiplied by PCR method.

To the target DNA fragment is attached an RI label or a non-RI label by a known method. The non-RI label is preferably utilized. Examples of the non-RI labels include fluorescence label, biotin label, and chemical luminescence label. The fluorescence label is most preferably employed. Examples of the fluorescence labels include cyanine dyes (e.g., Cy3 and Cy5 belonging to Cy Dye™ series), rhodamine 6G reagent, N-acetoxy-N²-acetylaminofluorene (AAF), and AAIF (iodide derivative of AAF). The target or sample DNA fragments labelled with different fluorescence indicators can be simultaneously analyzed, if the fluorescence indicators have fluorescence spectrum of different peaks. Also employable is an electroconductive label.

### Hybridization

The hybridization is performed by spotting an aqueous sample solution containing a target DNA fragment onto a DNA chip. The spotting is generally done in an amount of 1 to 100 nL. The hybridization is carried out by keeping the DNA chip having the spotted sample solution thereon at a temperature between room temperature and 70° C, for 6 to 20 hours. After the hybridization is complete, the DNA chip is washed with an aqueous buffer solution containing a surface active agent, to remove a free (namely, unfixed) sample DNA fragment. The surface active agent preferably is sodium dodecyl sulfate (SDS). The buffer solution may be a citrate buffer solution, a phosphate buffer solution, a borate buffer solution, Tris buffer solution, or Goods buffer solution. The citrate buffer solution is preferably employed.

The present invention is further described by the following examples.

### EXAMPLE 1

### Manufacture of Oligonucleotide-Fixed Plates Utilizing Irreversible Addition Reaction

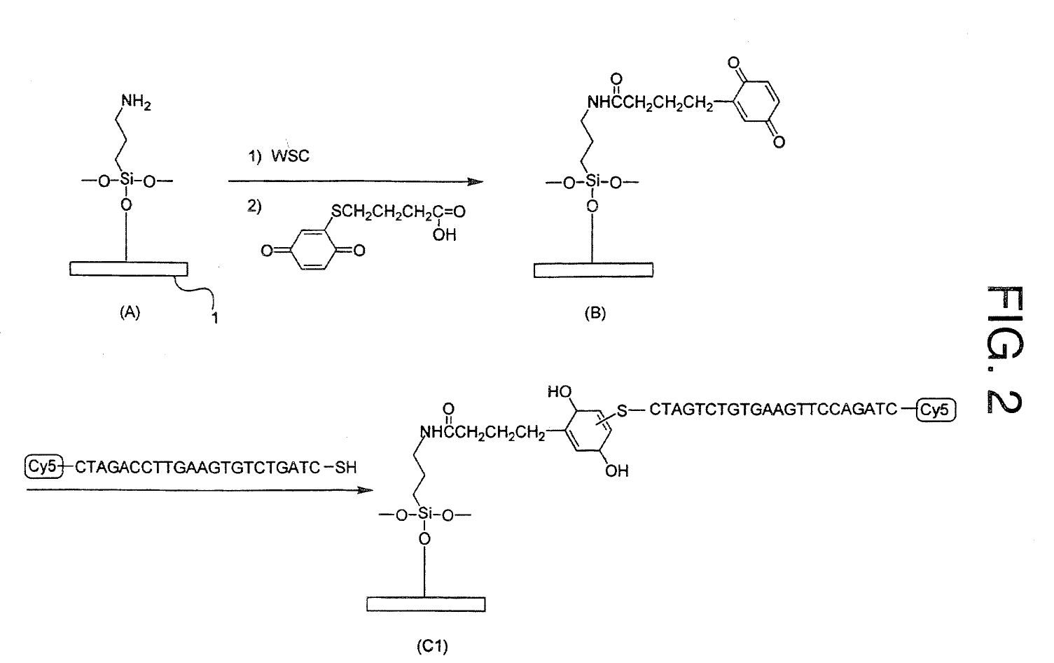

The reaction for the fixation is illustrated in the attached FIG. 2, in which 1 denotes a slide glass.

### (1) Preparation of Glass Plate (C1) Having on its Surface a Benzoquinone Moiety

A slide glass (25 mm×75 mm) was immersed in an ethanol solution of 2 wt. % aminopropylethoxysilane (available from Shin-etsu Chemical Industries, Co., Ltd.) for 10 minutes. Subsequently, the slide glass was taken out, washed with ethanol, and dried at 110° C. for 10 min. Thus, a silane coupling agent-treated slide glass (A) was prepared.

The silane coupling agent-treated slide glass (A) was then immersed in 50 mL of a solution of 2-(3-carboxypropyl)thio-1,4-benzoquinone (BQ, 2.5 g) and WSC (water-soluble carbodiimide, 955 mg) in acetonitrile for 2 hours. Subsequently, the slide glass was taken out of the solution, washed with acetonitrile, and dried for one hour under reduced pressure, to prepare a glass plate (B) having benzoquinone groups on its surface.

### (2) Fixation of Oligonucleotide and Measurement of Fluorescence Strength

An oligonucleotide (3′-CTAGTCTGTGAAGTGTCTGATC-5′, 22-mers) having a thiol group at 3′-terminal and a fluorescent label (FluoroLink, Cy 5-dCTP, available from Amasham Pharmacia Biotec Corp.) at 5′-terminal was dispersed in 1 μL of an aqueous solution containing a carbonate buffer solution (0.1 M, pH 9.3) at a concentration of 1×10⁻⁶M. The buffer solution was then spotted onto the glass plate (B) obtained in (1) above, and this was immediately kept at 60° C., 90% RH for one hour. Thus treated glass plate was then washed successively twice with a mixture of aqueous 0.1 wt. % SDS (sodium dodecyl sulfate) solution and aqueous 2×SSC solution (obtained by twice diluting standard sodium chloride-citrate buffer solution (SSC)), and once with the aqueous 0.2×SSC solution. Thus washed glass plate was placed in an aqueous 0.1 M thioglycol solution (pH 10) for 1.5 hours, washed with distilled water, and then dried at room temperature, to obtain a glass plate (C1) on which the oligonucleotides were fixed.

The fluorescence strength of thus treated plate (C1) was measured using a fluorescence scanning apparatus. The fluorescence strength was 1,680, which was well higher than the background fluorescence strength. This means that the oligonucleotides are well fixed onto the glass plate.

### EXAMPLE 2

### Detection of Target Oligonucleotide

### (1) Preparation of DNA Chip

A DNA chip, namely, glass plate (D2) on which the oligonucleotides were fixed was prepared in the same manner as in Example 1-(1) except for using the oligonucleotide having no fluorescent label.

### (2) Detection of Target Oligonucleotide

A target oligonucleotide (GATCAGACACTTCACAGACTAG-5′, 22-mers) having Cy5 (fluorescent label) at its 5′-terminal was dispersed in 20 μL of a hybridizing solution (mixture of 4×SSC and 10 wt. % SDS). The resulting solution was spotted onto the glass plate (D2) prepared in (1) above, and its spotted surface was covered with a covering glass. Thus covered chip was subjected to incubation at 60° C. for 20 hours in a moisture chamber. The incubated chip was washed successively with a mixture of 0.1 wt. % SDS and 2×SSC, a mixture of 0.1 wt. % SDS and 0.2×SSC, and an aqueous 0.2×SSC solution, centrifuged at 600 r.p.m. for 20 seconds, and dried at room temperature.

The fluorescence strength of thus treated glass plate was measured using a fluorescence scanning apparatus. The fluorescence strength was 632, which was well higher than the background fluorescence strength. This means that the target oligonucleotides are well fixed to the DNA chip having the complementary oligonucleotide probe.

### EXAMPLE 3

### Manufacture of Oligonucleotide-Fixed Plates Utilizing Cyclizing Addition Reaction

### (1) Preparation of Glass Plate (C) Having on its Surface a Maleimido Moiety (as Dienophile)

A slide glass (25 mm×75 mm) was immersed in an ethanol solution of 2 wt. % aminopropylethoxysilane (available from Shin-etsu Chemical Industries, Co., Ltd.) for 10 minutes. Subsequently, the slide glass was taken out, washed with ethanol, and dried at 110° C. for 10 min. Thus, a silane coupling agent-treated slide glass (A) was prepared.

The silane coupling agent-treated slide glass (A) was then immersed in an acetonitrile solution containing 5 wt. % maleic anhydride and 0.05 wt. % 4-dimethylaminopyridine for 10 minutes. Subsequently, the slide glass was taken out of the solution, washed with ethanol, and dried at 120° C. for 15 minutes, to prepare a glass plate (C) having maleimido groups on its surface.

### (2) Fixation of Oligonucleotide and Measurement of Fluorescence Strength

An oligonucleotide (3′-CTAGTCTGTGAAGTCTCGATC-5′, 22-mers) having an amino group at 3′-terminal and a fluorescent label (FluoroLink, Cy 5-dCTP, available from Amasham Pharmacia Biotec Corp.) at 5′-terminal was treated with furfural and sodium cyano horohydride, to prepare an probe compound having a furfuryl moiety at the 3′-terminal.

The probe compound was then dispersed in 1 μL of an aqueous solution containing a carbonate buffer solution (0.1 M, pH 9.8) at a concentration of 1×10⁻⁶M. The buffer solution was then spotted onto the glass plate (C) obtained in (1) above, and this was immediately kept at 60° C., 90% RH for one hour, and heated at 120° C. for 20 min. Thus treated glass plate was then washed successively twice with a mixture of aqueous 0.1 wt. % SDS (sodium dodecylsulfate) solution and aqueous 2×SSC solution (obtained by twice diluting standard sodium chloride-citrate buffer solution (SSC)), and once with the aqueous 0.2×SSC solution. Thus washed glass plate was dried at room temperature, to obtain a glass plate (D1) on which the oligonucleotides were fixed.

The fluorescence strength of thus treated plate (D1) was measured using a fluorescence scanning apparatus. The fluorescence strength was 1,599, which was well higher than the background fluorescence strength. This means that the oligonucleotides are well fixed onto the glass plate.

### EXAMPLE 4

### Detection of Target Oligonucleotide

### (1) Preparation of DNA Chip

A DNA chip, namely, glass plate (D2) on which the oligonucleotides were fixed was prepared in the same manner as in Example 3-(1) except for using the oligonucleotide having no fluorescent label.

### (2) Detection of Target Oligonucleotide

A target oligonucleotide (GATCAGACACTTCACAGACTAG-5′, 22-mers) having Cy5 (fluorescent label) at its 5′-terminal was dispersed in 20 μL of a hybridizing solution (mixture of 4×SSC and 10 wt. % SDS). The resulting solution was spotted onto the glass plate (D2) prepared in (1) above, and its spotted surface was covered with a covering glass. Thus covered chip was subjected to incubation at 60° C. for 20 hours in a moisture chamber. The incubated chip was washed successively with a mixture of 0.1 wt. % SDS and 2×SSC, a mixture of 0.1 wt. % SDS and 0.2×SSC, and an aqueous 0.2×SSC solution, centrifuged at 600 r.p.m. for 20 seconds, and dried at room temperature.

The fluorescence strength of thus treated glass plate was measured using a fluorescence scanning apparatus. The fluorescence strength was 625, which was well higher than the background fluorescence strength. This means that the target oligonucleotides are well fixed to the DNA chip having the complementary oligonucleotide probe.

### EXAMPLE 5

### Manufacture of Oligonucleotide-Fixed Plates Utilizing Coupling Reaction

### (1) Preparation of Glass Plate (C) Having on Its Surface a Benzoquinone Moiety

A slide glass (25 mm×75 mm) was immersed in an ethanol solution of 2 wt. % 1,2-bis(triethoxysilyl)ethane (silane coupling agent, available from Aldrich Corp.) for 10 minutes. Subsequently, the slide glass was taken out, washed with ethanol, and dried at 110° C. for 10 min. Thus, a silane coupling agent-treated slide glass (A) was prepared.

The silane coupling agent-treated slide glass (A) was then immersed in 50 mL of a solution of 4 wt. % diketene in acetonitrile for one hour. Subsequently, the slide glass was taken out of the solution, washed with acetonitrile, and dried for one hour under reduced pressure, to prepare a glass plate (C) having a diketene group on its surface.

### (2) Fixation of Oligonucleotide and Measurement of Fluorescence Strength

An oligonucleotide (3′-CTAGTCTGTGAAGTGTCTGATC-5′, 22-mers) having an amino group at 3′-terminal and a fluorescent label (FluoroLink, Cy 5-dCTP, available from Amasham Pharmacia Biotec Corp.) at 5′-terminal was dispersed in 1 μL of an aqueous solution containing a carbonate buffer solution (0.1 M, pH 9.3) at a concentration of 3×10⁻⁶M. To the resulting solution were added 1 μL of an aqueous solution of 1-ethyl-3-(3-dimethylaminopropyl)carbodiimide (9×10⁻⁶ M) and 1 μL of an aqueous solution of the compound having the below-illustrated formula (6×10⁻⁶ M):

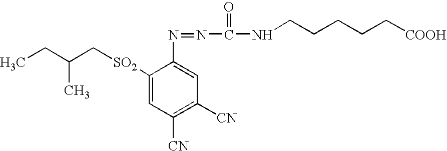

The resulting buffer solution was then spotted onto the glass plate (C) obtained in (1) above, and this was immediately kept at 25° C., 90% RH for one hour. Thus treated glass plate was then washed successively twice with a mixture of aqueous 0.1 wt. % SDS (sodium dodecyl sulfate) solution and aqueous 2×SSC solution (obtained by twice diluting standard sodium chloride-citrate buffer solution (SSC)), and once with the aqueous 0.2×SSC solution. Thus washed glass plate was placed in an aqueous 0.1 M glycine solution (pH 10) for 1.5 hours, washed with distilled water, and then dried at room temperature, to obtain a glass plate (D1) on which the oligonucleotides were fixed.

The fluorescence strength of thus treated plate (D1) was measured using a fluorescence scanning apparatus. The fluorescence strength was 1,555, which was well higher than the background fluorescence strength. This means that the oligonucleotides are well fixed onto the glass plate.

### EXAMPLE 6

### Detection of Target Oligonucleotide

### (1) Preparation of DNA Chip

A DNA chip, namely, glass plate (D2) on which the oligonucleotides were fixed was prepared in the same manner as in Example 5-(1) except for using the oligonucleotide having no fluorescent label.

### (2) Detection of Target Oligonucleotide

A target oligonucleotide (GATCAGACACTTCACAGACTAG-5′, 22-mers) having Cy5 (fluorescent label) at its 5′-terminal was dispersed in 20 μL of a hybridizing solution (mixture of 4×SSC and 10 wt. % SDS). The resulting solution was spotted onto the glass plate (D2) prepared in (1) above, and its spotted surface was covered with a covering glass. Thus covered chip was subjected to incubation at 60° C. for 20 hours in a moisture chamber. The incubated chip was washed successively with a mixture of 0.1 wt. % SDS and 2×SSC, a mixture of 0.1 wt. % SDS and 0.2×SSC, and an aqueous 0.2×SSC solution, centrifuged at 600 r.p.m. for 20 seconds, and dried at room temperature.

The fluorescence strength of thus treated glass plate was measured using a fluorescence scanning apparatus. The fluorescence strength was 580, which was well higher than the background fluorescence strength. This means that the target oligonucleotides are well fixed to the DNA chip having the complementary oligonucleotide probe.

What is claimed is:

1. An element comprising a solid carrier and a group of nucleotide derivatives or analogues thereof which are fixed to the solid carrier via covalent bonding produced by an irreversible addition reaction between a reactive group attached to the solid carrier and a reactive group attached to the nucleotide derivative or the analogue.

2. The element of claim 1, in which the reactive group attached to the solid carrier contains a quinone moiety and the reactive group attached to the nucleotide derivative or analogue contains an amino moiety or a thiol moiety.

3. The element of claim 2, in which the quinone moiety is a 1,4-benzophenone moiety or a 1,4-naphthoquinone moiety.

4. A method for preparing an element comprising a solid carrier and a group of nucleotide derivatives or their analogues fixed to the solid carrier via covalent bonding, which comprises bringing in a liquid phase a group of nucleotide derivatives or their analogues having at one end thereof or its vicinity a reactive group into contact with a solid carrier having thereon a reactive group, to cause an irreversible addition reaction between these reactive groups.

5. The method of claim 4, in which the reactive group attached to the solid carrier contains a quinone moiety and the reactive group attached to the nucleotide derivative or analogue contains an amino moiety or a thiol moiety.

6. The method of claim 5, in which the quinone moiety is a 1,4-benzophenone moiety or a 1,4-naphthoquinone moiety.

7. An element comprising a solid carrier and a group of nucleotide derivatives or analogues thereof which are fixed to the solid carrier via covalent bonding produced by a cyclizing addition reaction between a reactive group attached to the solid carrier and a reactive group attached to the nucleotide derivative or the analogue.

8. The element of claim 1, in which one of the reactive groups contains a diene moiety and another contains a dienophile moiety.

9. The element of claim 1, in which the reactive group attached to the nucleotide derivative or analogue contains a diene moiety and the reactive group attached to the solid carrier contains a dienophile moiety.

10. A method for preparing an element comprising a solid carrier and a group of nucleotide derivatives or their analogues fixed to the solid carrier via covalent bonding, which comprises bringing in a liquid phase a group of nucleotide derivatives or their analogues having at one end thereof or its vicinity a reactive group into contact with a solid carrier having thereon a reactive group, to cause a cyclizing addition reaction between these reactive groups.

11. The method of claim 10, in which one of the reactive groups contains a diene moiety and another contains a dienophile moiety.

12. The method of claim 10, in which the reactive group attached to the nucleotide derivative or analogue contains a diene moiety and the reactive group attached to the solid carrier contains a dienophile moiety.

13. An element comprising a solid carrier and a group of nucleotide derivatives or analogues thereof which are fixed to the solid carrier via covalent bonding produced by a coupling reaction between a reactive group attached to the solid carrier and a reactive group attached to the nucleotide derivative or the analogue.

14. An element of claim 13, in which one reactive group is derived from a compound having the following formula (1):
Z=(CR¹-CR²)ₙ═N—R³ (1)
wherein Z is an oxygen atom or a group of R⁴N or R⁴K⁵N⁺Ain which each of R⁴ and R⁵ independently is a hydrogen atom, an alkyl group or an aryl group, and A is an inorganic or organic anion; each of R¹ and R² independently is a hydrogen atom or a substituent group; R³ is a hydrogen atom, or a group of NR⁶R⁷, SR⁶, SO₂R⁶, or COR⁶ in which each of R⁶ and R⁷ independently is a hydrogen atom or a substituent group; n is 0, 1, or 2; provided that R¹ and R² can be combined together to form a ring; and at least one of Z, R¹ and R² produces covalent bonding with a surface of the solid carrier or the nucleotide derivative or analogue; and another reactive group is capable of causing a coupling reaction with a nitrogen atom of the reactive group derived from the compound of the formula (1).

15. The element of claim 14, wherein the reactive group derived from the formula (1) is attached to the surface of the solid carrier, and the reactive group causing a coupling reaction with a nitrogen atom of the reactive group derived from the formula (1) is attached to the nucleotide derivative or analogue.

16. A method for preparing an element comprising a solid carrier and a group of nucleotide derivatives or their analogues fixed to the solid carrier via covalent bonding, which comprises bringing in a liquid phase a group of nucleotide derivatives or their analogues having at one end thereof or its vicinity a reactive group into contact with a solid carrier having thereon a reactive group, to cause a coupling reaction.

17. The method of claim 16, in which one reactive group is derived from a compound having the following formula (1):
Z=(CR¹-CR²)ₙ═N—R³ (1)
wherein Z is an oxygen atom or a group of R⁴N or R⁴R⁵N⁺Ain which each of R⁴ and R⁵ independently is a hydrogen atom, an alkyl group or an aryl group, and A is an inorganic or organic anion; each of R¹ and R² independently is a hydrogen atom or a substituent group; R³ is a hydrogen atom, or a group of NR⁶R⁷, SR⁶, SO₂R⁶, or COR⁶ in which each of R⁶ and R⁷ independently is a hydrogen atom or a substituent group; n is 0, 1, or 2; provided that R¹ and R² can be combined together to form a ring; and at least one of Z, R¹ and R² produces covalent bonding with a surface of the solid carrier or the nucleotide derivative or analogue; and another reactive group is capable of causing a coupling reaction with a nitrogen atom of the reactive group derived from the compound of the formula (1).

18. The method of claim 17, wherein the reactive group derived from the formula (1) is attached to the surface of the solid carrier, and the reactive group causing a coupling reaction with a nitrogen atom of the reactive group derived from the formula (1) is attached to the nucleotide derivative or analogue.
